(19) United States
(12) Patent Application Publication
(10) Pub. No.: US 2009/0118512 A1
(43) Pub. Date: May 7, 2009

(54) Process for Preparing 6,7-Dihydro-5H-Imidazo[1,5-A]Pyridin-8-One

(76) Inventors: Pierre Martin, Rheinfelden (CH); Andreas Boudier, Basel (CH); Michael Quirmbach, Basel (CH); Robert Mah, Allschwil (CH); Nathalie Jotterand, Allschwil (CH)

Correspondence Address: WENDEROTH, LIND & PONACK, L.L.P., 1030 15th Street, N.W.,, Suite 400 East, Washington, DC, 20005-1503, US

(21) Appl. No.: 12/225,813

(22) PCT Filed: Mar. 30, 2007
(86) PCT No.: PCT/EP2007/053078

§ 371 Date: Sep. 30, 2008

(30) Foreign Application Priority Data
Mar. 31, 2006 (CH) 00535/06

(51) Int. Cl. C07D 471/04; C07D 211/74; C07F 7/10; C07D 233/64

(52) U.S. Cl. 546/121; 546/300; 548/110; 548/333.5

(57) ABSTRACT

6,7-Dihydro-5H-imidazo[1,5-a]pyridin-8-one (I), is obtainable in high yields by: 1) a process which proceeds from a suitably protected C-(3-hydroxypyridin-2-yl)methylamine whose amine is converted to the formamide which is then cyclized to the imidazo[1,5-a]pyridine and hydrogenated to the 6,7-dihydro-5H-imidazo[1,5-a]pyridin-8-one, and suitably protected C-(3-hydroxypyridin-2-yl)methylamines can be prepared either in 2 steps proceeding from commercially available 3-hydroxy-2-cyanopyridine [932-35-4] or in 3 steps proceeding from commercially available 2-hydroxymethylpyridin-3-ol [14173-30-9]; 2) a process for preparing 4-hydroxy-1-(1H-imidazol-4-yl)butan-1-one, an intermediate from the synthesis of 6,7-dihydro-5H-imidazo[1,5-a]pyridin-8-one (formula I), as described in WO 2002/040484, proceeding from N,N-dimethyl-2-(trialkylsilanyl)imidazole-1-sulphonamide by lithiation and subsequent reaction with a suitably protected 4-hydroxybutyraldehyde, followed by oxidation of the secondary alcohol, acid-induced deprotection of the imidazole and deprotection of the alcohol functionality; 3) a process which proceeds from 5,6,7,8-tetrahydroimidazo[1,5-a]pyridine [38666-30-7], which is oxidized.

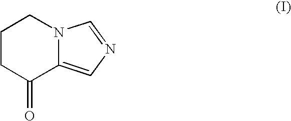

### FIELD OF THE INVENTION

The invention relates to novel processes for preparing 6,7-dihydro-5H-imidazo[1,5-a]pyridin-8-one and to novel intermediates which are obtained in the process steps.

### BACKGROUND OF THE INVENTION

WO 2005/118581 describes 8-substituted 5,6,7,8-tetrahydroimidazo[1,5-a]pyridines which have aldosterone synthase-inhibiting properties and can be used in pharmaceutical formulations as a human medicine for the prevention, for retarding the progression of or for treating pathological states which are caused completely or partly by hyperaldosteronism. The preparation processes described there use 6,7-dihydro-5H-imidazo[1,5-a]pyridin-8-one [51907-18-7] as the starting material, which is prepared by the synthesis described in WO2002/040484. In the synthesis described there, 6,7-dihydro-5H-imidazo[1,5-a]pyridin-8-one is obtainable in 5 steps starting from 4-(tetrahydro-2H-pyran-2-yloxy)-1-(1-trityl-1H-imidazol-4-yl)-2-butyn-1-one, which can be prepared in 2 steps proceeding from commercially available 1-trityl-1H-imidazole-4-carboxylic acid [191103-80-7] after conversion to the corresponding Weinreb amide by addition of the lithium salt of 2-prop-2-ynyloxy-3,4-dihydro-2H-pyran. This synthesis, which thus has 7 steps, for the preparation of 6,7-dihydro-5H-imidazo[1,5-a]pyridin-8-one is unsuitable for an industrial process especially with regard to the yields, some of which are unsatisfactory. The addition of lithium species to the Weinreb amide is notable in particular for poor yields. The last two synthesis steps, proceeding from 4-hydroxy-1-(1-trityl-1H-imidazol-4-yl)butan-1-one by reaction with mesyl chloride and subsequent ring closure in a one-pot process, also afford the 6,7-dihydro-5H-imidazo[1,5-a]pyridin-8-one in very poor yields.

### DETAILED DESCRIPTION OF THE INVENTION

In the process according to the invention which is now proposed, the 4-hydroxy-1-(1H-imidazol-4-yl)butan-1-one, an intermediate from the synthesis as described in WO2002/040484, is therefore prepared proceeding from N,N-dimethyl-2-(trialkylsilanyl)imidazole-1-sulphonamide by lithiation and subsequent reaction with a suitably protected 4-hydroxybutyraldehyde, followed by oxidation of the secondary alcohol, acid-induced deprotection of the imidazole and deprotection of the alcohol functionality. From this synthesis route, the longest linear sequence to the preparation of 6,7-dihydro-5H-imidazo[1,5-a]pyridin-8-one proceeding from commercially available starting material is, depending on the choice of the protection group on the alcohol functionality, either equally long or, with 8 steps, one step longer, but the first 5 steps all proceed with particularly high yields.

Additionally proposed in accordance with the invention is a process which proceeds from a suitably protected C-(3-hydroxypyridin-2-yl)methylamine whose amine is converted to the formamide which is then cyclized to the imidazo[1,5-a]pyridine and hydrogenated to the 6,7-dihydro-5H-imidazo[1,5-a]pyridin-8-one. It has been found that suitably protected C-(3-hydroxypyridin-2-yl)methylamines can be prepared in 2 steps proceeding from commercially available 3-hydroxy-2-cyanopyridine [932-35-4]. Suitably protected C-(3-hydroxypyridin-2-yl)methylamines are also obtainable in 3 steps proceeding from commercially available 2-hydroxymethylpyridin-3-ol [14173-30-9] via the synthesis described in U.S. Pat. No. 4,409,226. In this novel process, the 6,7-dihydro-5H-imidazo[1,5-a]pyridin-8-one is obtained in 6 or 7 synthesis steps proceeding from commercially available starting material. The synthesis steps are notable in that all steps proceed reproducibly with high yields, this synthesis route needs far less protecting group manipulation and has higher atom economy, and in that it is possible to largely dispense with complicated purification by chromatography processes, which means a considerable advantage (for example cost saving) for the production on the industrial scale.

Furthermore, a process is proposed in accordance with the invention which proceeds from commercially available 5,6,7,8-tetrahydroimidazo[1,5-a]pyridine [38666-30-7] which is oxidized directly to 6,7-dihydro-5H-imidazo[1,5-a]pyridin-8-one. In this novel process, the 6,7-dihydro-5H-imidazo[1,5-a]pyridin-8-one is obtained in only 1 synthesis step proceeding from commercially available starting material. This process is notable in that the synthesis route leads particularly rapidly to the target molecule, which means a considerable advantage (for example cost saving) for production on the industrial scale.

The invention provides a process for preparing 6,7-dihydro-5H-imidazo[1,5-a]pyridin-8-one (formula I)

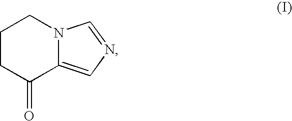

characterized in that
1a) an aldehyde of the formula II

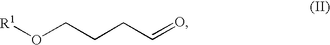

in which R¹ is a suitable protecting group which is either orthogonal to the other protecting groups used in the process, for example benzyl or 4-methoxybenzyl, or which can be concomitantly removed, for example a trialkylsilyl group,
is reacted with N,N-dimethyl-2-(trialkylsilanyl)imidazole-1-sulphonamide (formula III)

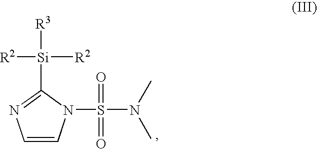

in which R² and R³ are each independently methyl, ethyl, isopropyl, tert-butyl or isobutyl, or where R³ is additionally also —C(CH₃)₂—CH(CH₃)₂, to give a compound of the formula IV

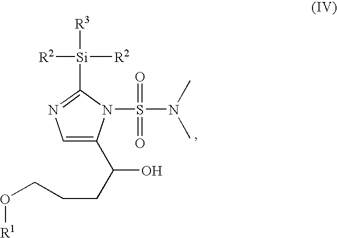

in which R¹, R² and R³ are each as defined above,
1b) the OH group of the compound of the formula IV is oxidized to a ketone to thus obtain a compound of the formula V

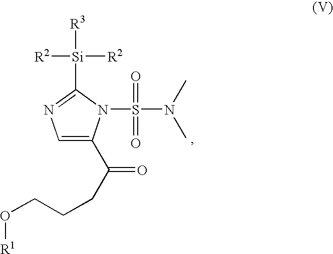

in which R¹, R² and R³ are each as defined above,
1c) the protecting groups on the imidazole of the compound of the formula V are removed to obtain a compound of the formula VI

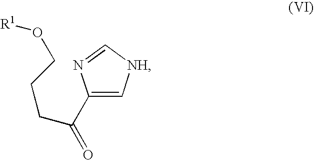

in which
R¹ is a suitable protecting group orthogonal to the other protecting groups used in the process, for example benzyl or 4-methoxybenzyl,
1d) the protecting group of the alcohol function of the compound of the formula VI is removed to thus obtain 4-hydroxy-1-(1H-imidazol-4-yl)butan-1-one (formula VII)

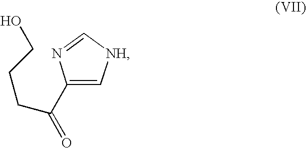

whereby step 1d) is obsolete in case the protecting group R¹ is chosen to be a protecting group which can be concomitantly removed together with the protecting groups on the imidazole moiety as 4-hydroxy-1-(1H-imidazol-4-yl)butan-1-one (formula VII) is obtained as the product in step 1c), and
1e) the alcohol function of the 4-hydroxy-1-(1H-imidazol-4-yl)butan-1-one (formula VII) is converted to a leaving group followed by the ring closure to 6,7-dihydro-5H-imidazo[1,5-a]pyridin-8-one (formula I).

The invention further provides a process for preparing 6,7-dihydro-5H-imidazo[1,5-a]pyridin-8-one (formula I)

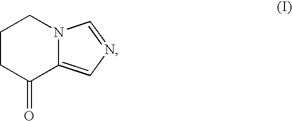

which is characterized in that
2a) a compound of the formula VIII

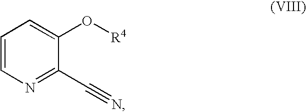

where R⁴ is a suitable protecting group removable by hydrogenation under certain conditions, for example benzyl or 4-methoxybenzyl,
is selectively hydrogenated at the cyano functionality to a compound of the formula IX
or
2a′) 2-hydroxymethylpyridin-3-ol hydrochloride (formula (VIII′)

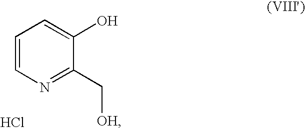

is converted by selective phase transfer-catalysed O-alkylation of the alcohol in the 3 position with an R⁴-halide, followed by mesylation of the primary alcohol, reaction with potassium phthalimide and subsequent treatment with aqueous dimethylamine solution, preferably 40% aqueous dimethylamine solution, to a compound of the formula IX,
2b) the compound of the formula IX

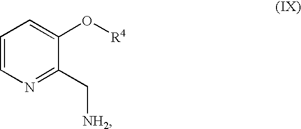

is converted by reaction with formic acid to give the compound of the formula X

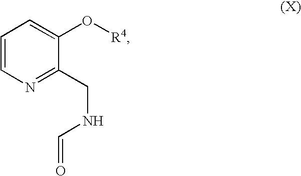

2c) the compound of the formula X is converted to the compound of the formula XI

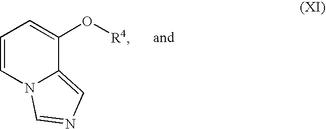

2d) the compound of the formula XI is hydrogenated to obtain 6,7-dihydro-5H-imidazo[1,5-a]pyridin-8-one (formula I).

The invention additionally provides a process for preparing 6,7-dihydro-5H-imidazo[1,5-a]pyridin-8-one (formula I)

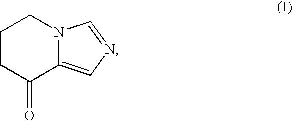

which is characterized in that
3a) 5,6,7,8-tetrahydroimidazo[1,5-a]pyridine [38666-30-7] (formula XII)

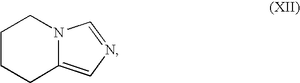

is oxidized regioselectively to obtain 6,7-dihydro-5H-imidazo[1,5-a]pyridin-8-one (formula I).

The starting compounds of the formulae II and III used in process step 1a) are known and can be prepared by known processes. For example, 4-benzyloxybutyraldehyde (formula II, R¹=benzyl) or 4-[[(1,1-dimethylethyl)dimethylsilyl]oxy]-1-butyraldehyde (R1=[(1,1-dimethylethyl)dimethylsilyl) can be prepared by oxidation of commercially available 4-benzyloxybutan-1-ol [4541-14-4] or 4-[[(1,1-dimethylethyl)dimethylsilyl]oxy]-1-butanol [87184-99-4], respectively according to the process described by C. Dardonville and I. H. Gilbert in Organic & Biomolecular Chemistry Vol. 1(3), (2003), pages 552-559. N,N-Dimethyl-2-(trialkylsilanyl)imidazole-1-sulphonamide (formula II) is prepared, for example, by the process described by Y. Lee, P. Martasek, L. J. Roman, B. S. S. Masters and R. B. Silverman in Bioorganic & Medicinal Chemistry, Vol. 7(9), (1999), pages 1941-1951, by lithiation of commercially available N,N-dimethyl-imidazole-1-sulphonamide [78162-58-0] and sub-sequent reaction with trialkylchlorosilane.

The reaction of process step 1a) is obtained in analogy to known processes, for example to the process published by A. Frankowski in Tetrahedron Vol. 59(34), (2003), pages 6503-6520. The reaction is performed advantageously at temperatures between −78° C. and −40° C. in the presence of at least equivalent amounts of a strong base. Suitable bases are particularly alkali metal lower alkyls, for example n-, sec- or tert-butyllithium, or lithiated amines, for example lithium diisopropylamide. It is appropriate to use the imidazole component in a slight excess of 1.05 to 1.2 molar equivalents. The reaction is also appropriately performed in a solvent, for which ethers, for example diethyl ether, tetrahydrofuran and dioxane, are particularly suitable. The secondary alcohol of the formula IV is obtained in yields over 80%.

The oxidation of the OH group of the secondary alcohol of the formula IV in process step 1b) is performed in analogy to known processes, for example to the process published by R. Leurs in Journal of Medicinal Chemistry, Vol. 46(25), (2003), pages 5445-5457, or to the process published by R. E. Boyd in Journal of Medicinal Chemistry, Vol. 44(6), (2001), pages 863-872. Particularly suitable methods are the classical Swern oxidation using oxalyl chloride in the presence of dimethyl sulphoxide and triethylamine, which is performed in this case advantageously at temperatures between −78° C. and −40° C., in the presence of a suitable nonpolar solvent, for example dichloromethane or the oxidation using manganese dioxide, which is performed advantageously at temperatures between 15° C. and 40° C., in the presence of a suitable nonpolar solvent, for example dichloromethane. The ketone of the formula V is obtained in yields over 80% and advantageously used in the next step without purification.

The removal of the protecting groups on the imidazole of the ketone of the formula V in process step 1c) is obtained in analogy to known processes, for example to the process published by A. Frankowski in Tetrahedron, Vol. 59(34), (2003), pages 6503-6520, by treatment with hydrochloric acid. The reaction is performed advantageously at relatively high temperatures, for example 30-80° C., in the presence of a 2-5M aqueous hydrochloric acid solution in a water-miscible solvent, for example tetrahydrofuran. The ketone of the formula VI is obtained in yields over 70% and advantageously used in the next step without purification.

The removal of the benzyl or 4-methoxybenzyl protecting group of the ketone of the formula VI in process step 1d) is known in principle and can be performed in analogy to known processes, for example to Frankowski in Tetrahedron Vol. 59(34), (2003), pages 6503-6520 by hydrogenation. The hydrogenation of the benzyl or 4-methoxybenzyl or 4-methoxybenzyl protecting group of the ketone of the formula VI in process step 1d) is advantageously performed with Pd(OH)₂/C (Pearlman's catalyst) in acidic medium, for example in ethanolic HCl, or a mixture of an alcohol, for example ethanol, methanol, butanol, with acetic acid at temperatures of 10-60° C. under an atmosphere of hydrogen (advantageously under standard pressure). The 4-hydroxy-1-(1H-imidazol-4-yl)butan-1-one (formula VII) is obtained in yields over 90% and in high purity, possibly as the hydrochloride. Chromatographic purification of the compound is not required.

The ring closure of 4-hydroxy-1-(1H-imidazol-4-yl)butan-1-one (formula VII) of process step 1e) is known per se and can be performed by the process described in WO 2002/040484. To this end, the alcohol function is first converted to a leaving group, for example to a mesylate or tosylate. This reaction step is performed using methanesulphonyl chloride or toluenesulphonyl chloride in an apolar solvent, for example in a chlorinated solvent such as dichloromethane, using an amine base, for example triethylamine, at temperatures between 10° C. and 25° C. The sulphonic esters thus obtained are closed by provision of energy, for example by the action of heat to give 6,7-dihydro-5H-imidazo[1,5-a]pyridin-8-one (formula I). The ring closure step is performed in a high-boiling (boiling point over 75° C.) polar solvent, for example in acetonitrile.

The starting compounds of the formula VIII used in process step 2a) are known and can be prepared by known processes. For example, 3-benzyloxypyridine-2-carbonitrile [24059-90-3] (formula VIII, R⁴=benzyl) is known and can be prepared by benzylating commercially available 3-hydroxypyridine-2-carbonitrile [932-354] according to the process described in WO02/044153.

The hydrogenation of the cyano group of the compound of the formula VIII in process step 2a) is performed in analogy to known processes, for example to the process published by M. B. Young in Journal of Medicinal Chemistry, Vol. 47(12), (2004), pages 2995-3008. Suitable catalysts for this selective transformation are, for example, nickel catalysts; for example, the reduction can be performed using Raney nickel as the catalyst. The reaction is advantageously performed in alcoholic solvents, if appropriate with an additive. Suitable additives are bases, for example ammonia, or amine bases, for example ethanolamine. The reaction is performed advantageously at temperatures of room temperature to 80° C. under standard pressure or elevated pressure of hydrogen, for example 1-80 bar. The protected C-(3-hydroxypyridin-2-yl)methylamine of the formula IX is obtained in yields of 91% in sufficient purity. Chromatographic purification of the compound is not required.

The conversion of the 2-hydroxymethylpyridin-3-ol hydrochloride [14173-30-9] (formula VIII′) in process step 2a′) to the protected C-(3-hydroxypyridin-2-yl)-methylamine of the formula IX is known and can be performed by the process described in U.S. Pat. No. 4,409,226.

The transformation of the protected C-(3-hydroxypyridin-2-yl)methylamine of the formula IX to the protected N-(3-hydroxypyridin-2-ylmethyl)formamide of the formula X by reaction with formic acid in process step 2b) is performed in analogy to known processes, for example to the process published by L. J. Browne in Journal of Medicinal Chemistry, Vol. 34(2), (1991), pages 725-736. The reaction is advantageously preformed using formic acid as the solvent, or in a high-boiling (boiling point over 75° C.) solvent, for example in toluene, at temperatures between 40° C. and 100° C. The protected N-(3-hydroxypyridin-2-ylmethyl)formamide of the formula X is obtained in yields over 95% and advantageously used without purification in the next step.

The ring closure of the protected N-(3-hydroxypyridin-2-ylmethyl)formamide of the formula X in process step 2c) by treatment with phosphoryl chloride is performed in analogy to known processes, for example to the process published by L. J. Browne in Journal of Medicinal Chemistry, Vol. 34(2), (1991), pages 725-736. The reaction is performed advantageously using a high-boiling (boiling point over 75° C.) solvent, for example toluene, at temperatures between 80° C. and 115° C. The protected 8-hydroxyimidazo[1,5-a]pyridine of the formula XI is obtained in yields over 65%.

The removal of the benzyl protecting group with simultaneous partial reduction of the pyridine structure of the protected 8-hydroxyimidazo[1,5-a]pyridine of the formula XI in process step 2d) is advantageously performed by hydrogenation using a heterogeneous hydrogenation catalyst, for example palladium on carbon. The reaction is advantageously performed in an alcoholic solvent, for example methanol, at temperatures of 20° C.-60° C. under elevated pressure of hydrogen, for example 1.2-20 bar. The 6,7-dihydro-5H-imidazo[1,5-a]pyridin-8-one (formula I) is obtained in yields of 75% in high purity. Chromatographic purification of the compound is not required.

The oxidation of the 5,6,7,8-tetrahydroimidazo[1,5-a]pyridine [38666-30-7] (formula XII) in process step 3a) is performed advantageously using potassium permanganate as the oxidizing agent. The reaction is performed appropriately in a polar aprotic solvent, for example in acetonitrile and at temperatures between 15° C. and 40° C. The 6,7-dihydro-5H-imidazo[1,5-a]pyridin-8-one (formula I) is obtained in yields of 70%. The purity is sufficient for the further use of the compound (90% according to NMR).

The process according to the invention for preparing 6,7-dihydro-5H-imidazo[1,5-a]pyridin-8-one (formula I) makes it possible to prepare the intermediates over all process steps in high yields. The high overall yields make the process suitable for industrial use.

The invention also provides the following compounds (intermediates):

protected N,N-dimethyl-5-(4-hydroxy-1-hydroxybutyl)-2-(trialkylsilanyl)imidazole-1-sulphonamide of the formula IV

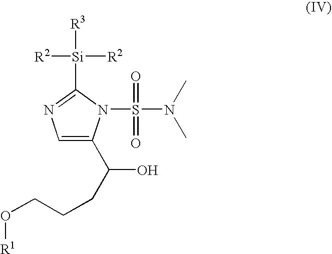

in which
R¹ is a suitable protecting group which is either orthogonal to the other protecting groups used in the process, for example benzyl or 4-methoxybenzyl, or which can be concomitantly removed, for example a trialkylsilyl group,
R² and R³ are each independently methyl, ethyl, isopropyl, tert-butyl or isobutyl or where R³ is additionally also —C(CH₃)₂—CH(CH₃)₂,
protected N,N-dimethyl-5-(4-hydroxybutyryl)-2-(trialkylsilanyl)imidazole-1-sulphonamide of the formula V

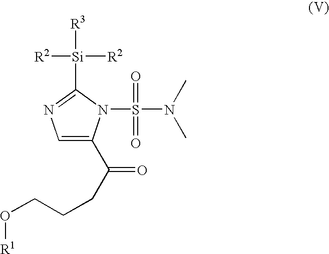

in which R¹, R² and R³ assume the above-specified definitions, and
protected 4-hydroxy-1-(1H-imidazol-4-yl)butan-1-one of the formula VI

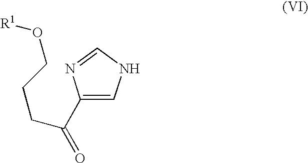

in which R¹ assumes the above-specified definitions.

The invention further provides the following compounds (intermediates):

protected N-(3-hydroxypyridin-2-ylmethyl)formamide of the formula X

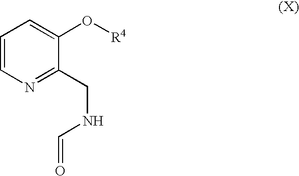

in which R⁴ assumes the above-specified definitions, and
protected 8-benzyloxyimidazo[1,5-a]pyridine of the formula XI

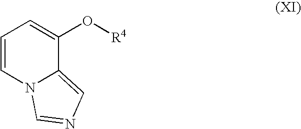

in which R⁴ assumes the above-specified definitions.

In the context of the present invention alkyl denotes linear or branched radicals, preferably C₁-C₈-alkyl and most preferred C₁-C₄-alkyl, for example methyl, ethyl, propyl, isopropyl, butyl and ter-butyl.

The references given above set out analogous reaction conditions and are incorporated by reference herein in their entirety.

Numbers given in square brackets refer to Chemical Abstracts registry numbers.

The examples which follow illustrate the invention in detail.

### EXAMPLES

HPLC gradients on Hypersil BDS C-18 (5 μm); column 4×125 mm

95% water*/5% acetonitrile* to 0% water*/100% acetonitrile* in 10 minutes+2 minutes (1 ml/min)

* contains 0.1% trifluoroacetic acid

The following abbreviations are used:
Rf ratio of distance traveled by a substance to separation of the eluent front from the start point in thin-layer chromatography
Rt retention time of a substance in HPLC (in minutes)
m.p. melting point (temperature)

### Example 1A

### Process for Preparing N,N-dimethyl-5-(4-benzyloxy-1-hydroxybutyl)-2-(tert-butyldimethylsilanyl)imidazole-1-sulphonamide (Formula IV)

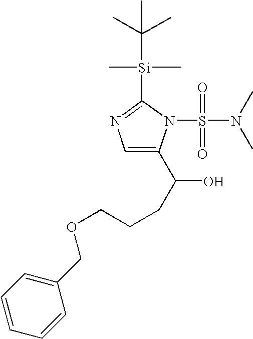

A solution of 186.000 mmol of N,N-dimethyl-2-(tert-butyldimethylsilanyl)imidazole-1-sulphonamide [129378-52-5] in 1.35 l of tetrahydrofuran is cooled to internal temperature approx. −70° C. by means of an external ethanol/dry ice bath. A solution of 203.000 mmol of sec-butyllithium (156 ml, 1.3 M in cyclohexane) is added dropwise within 45 minutes. The reaction mixture is stirred at this temperature for a further 30 minutes. A solution of 169.000 mmol of 4-benzyloxybutyraldehyde in 150 ml of tetrahydrofuran is then added dropwise with stirring within 30 minutes. The mixture is stirred at −70° C. for a further hour, the cold bath is removed and the reaction mixture is quenched at 0° C. cautiously with 200 ml of aqueous saturated ammonium chloride solution, followed by 200 ml of water. The organic phase is removed and the aqueous phase is then extracted with tert-butyl methyl ether (2×200 ml). The combined organic phases are washed with brine (1 l), dried over magnesium sulphate and concentrated by evaporation. 141.111 mmol (83% based on the aldehyde) of the title compound are obtained from the residue as a brownish oil via flash chromatography (SiO₂ 60F, heptane/ethyl acetate=1.5:1). Rf=0.68 (heptane/ethyl acetate=1:2), Rt=7.96.

### Example 1B

### Process for Preparing N,N-diemthyl-5-(4-benzyloxybutyryl)-2-(tert-butyldimethylsilanyl)imidazole-1-sulphonamide (Formula V)

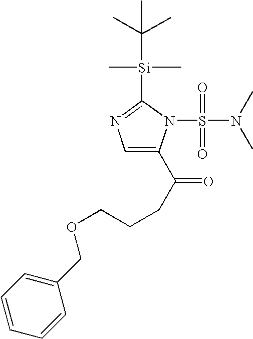

A baked-out apparatus under argon protective gas is initially charged with 256.000 mmol of oxalyl chloride in 500 ml of dichloromethane and cooled to internal temperature approx. —70° C. by means of an external ethanol/dry ice bath. A solution of 512.000 mmol of dimethyl sulphoxide in 100 ml of dichloromethane is added dropwise such that the internal temperature is below −60° C. (Caution: vigorous CO₂ evolution!). The reaction mixture is stirred for a further 5 minutes, then a solution of 128.000 mmol of N,N-dimethyl-5-(4-benzyloxy-1-hydroxybutyl)-2-(tert-butyldimethylsilanyl)imidazole-1-sulphonamide (Example 1A) in 150 ml of dichloromethane is added dropwise within 20 minutes. The reaction mixture is stirred at approx. −65° C. for 15 minutes and then admixed with 640.000 mmol of triethylamine. The cold bath is removed and the reaction mixture is warmed slowly to room temperature. The reaction mixture is admixed with 500 ml of aqueous saturated sodium bicarbonate solution with stirring, the phases are separated and the aqueous phase is then washed with dichloromethane (2×500 ml). The combined organic phases are dried over sodium sulphate and concentrated by evaporation. 104.361 mmol (81%) of the title compound are obtained from the residue as an amber-coloured liquid via flash chromatography (SiO₂ 60F, heptane/ethyl acetate=2:1). Rf=0.81 (heptane/ethyl acetate=1:2), Rt=8.00.

### Example 1C

### Process for Preparing 4-benzyloxy-1-(1H-imidazol-4-yl)-butan-1-one (Formula VI)

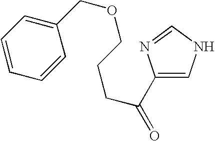

A solution of 103.000 mmol of N,N-dimethyl-5-(4-benzyloxybutyryl)-2-(tert-butyldimethylsilanyl)imidazole-1-sulphonamide (Example 1B) in 200 ml of tetrahydrofuran is admixed with 200 ml of aqueous 4 M hydrochloric acid and stirred at 60° C. for 3 hours. The reaction mixture is cooled and washed with diethyl ether (2×200 ml). The ethereal wash phase is discarded. The aqueous phase is basified with 4 M sodium hydroxide solution (pH 10) and extracted repeatedly with ethyl acetate (3×200 ml). The combined ethyl acetate phases are washed with brine (1×400 ml), dried over sodium sulphate and concentrated by evaporation. The residue is digested in diethyl ether and filtered. 73.300 mmol (73%) of the title compound are obtained as a beige solid. Rf=0.38 (dichloromethane:methanol=90:10), Rt=5.01.

### Example 1D

### Process for Preparing 4-hydroxy-1-(1H-imidazol-4-yl)butan-1-one hydrochloride (Formula VII)

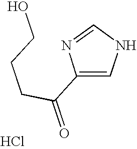

A solution of 64.700 mmol of 4-benzyloxy-1-(3H-imidazol-4-yl)butan-1-one (Example 1C) in 150 ml of ethanol and 34 ml of 4 M ethanolic HCl is hydrogenated with 8 g of Pearlman's catalyst (Pd(OH)₂/C) (Fluka 76063, Batch: 453975/1) in a hydrogenation apparatus at room temperature and under hydrogen under standard pressure for 5 hours. An addition of 4 g of Pd(OH)₂/C and a further 2 hours of hydrogenation are required to obtain complete conversion. The apparatus is decompressed and flushed with nitrogen. The reaction mixture is filtered off through Hyflo [91053-39-3] (Hyflo Super Cel medium, Fluka 76063), and the filtrate is concentrated by evaporation. The residue is then concentrated by evaporation repeatedly with acetonitrile (2×50 ml) and then dried under high vacuum. 59.300 mmol (92%) of the title compound are then obtained from the residue as a beige solid. The substance is identical to the already published material from WO 2002/040484.

### Example 2A

### Process for Preparing C-(3-benzyloxypyridin-2-yl)methylamine [6059-29-6] (Formula IX)

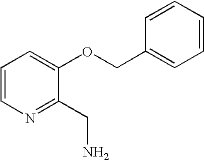

An autoclave is charged with 200 ml of methanolic ammonia solution (13% w/w in methanol), 120.000 mmol of 3-benzyloxypyridine-2-carbonitrile [24059-90-3] and 5.0 g of RaNi Actimed M (Engelhard, Code: 3799.4, Lot 2012.28026). The mixture is hydrogenated at 60 bar of hydrogen pressure and 60° C. for 7 hours. The heater is switched off, the reaction mixture is clarified by filtration through Hyflo and the filtercake is washed with methanol. The filtrate is concentrated by evaporation. 109.675 mmol (91% crude yield) of the title compound are obtained from the residue as a black liquid. The material is used in the next step without further purification. The analysis agrees with published data. ¹H NMR, m/e=215 [M+H]⁺

### Example 2B

### Process for Preparing N-(3-benzyloxypyridin-2-ylmethyl)formamide (Formula X)

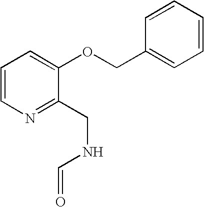

A solution of 52.300 mmol of C-(3-benzyloxypyridin-2-yl)methylamine [88423-14-7] (Example 2A) in 35 ml of formic acid is heated to reflux for 3 hours. The reaction mixture is cooled and adjusted cautiously to pH 8 with approx. 100 ml of ammonium hydroxide solution (25% in water) with ice bath cooling. The mixture is diluted with 100 ml of water and the aqueous phase is extracted repeatedly with dichloromethane (3×250 ml). The combined organic phases are dried over sodium sulphate and concentrated by evaporation. 51.590 mmol (99% crude yield) of the title compound are obtained from the residue as a brown oil. The material is used in the next step without further purification. Rf=0.16 (ethyl acetate), Rt=4.58.

### Example 2C

### Process for Preparing 8-benzyloxyimidazo[1,5-a]pyridine (Formula XI)

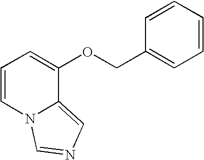

A solution of 51.590 mmol of N-(3-benzyloxypyridin-2-ylmethyl)formamide (Example 2B) in 250 ml of toluene is admixed with 87.700 mmol of phosphoryl chloride. The reaction mixture is heated to reflux over 1.5 hours. The reaction mixture is cooled and adjusted cautiously to pH 8 with approx. 30 ml of ammonium hydroxide solution (25% in water) with ice bath cooling. The mixture is diluted with 200 ml of water and the aqueous phase is extracted repeatedly with ethyl acetate (3×150 ml). The combined organic phases are dried over sodium sulphate and concentrated by evaporation. 34.424 mmol (67%) of the title compound are obtained from the residue as a light brown resin by means of flash chromatography (SiO₂ 60F, ethyl acetate). Rf=0.38 (ethyl acetate), Rt=5.42.

### Example 2D

### Process for Preparing 6,7-dihydro-5H-imidazo[1,5-a]pyridin-8-one (Formula I)

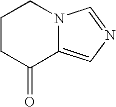

A solution of 34.300 mmol of 8-benzyloxyimidazo[1,5-a]pyridine (Example 2C) in 80 ml of methanol is hydrogenated with 3.85 g of 5% Pd/C (Engelhard 4522, Batch: 390680) in a Parr apparatus at room temperature and 4 bar of hydrogen for 20 hours. Addition of 3.85 g of 5% Pd/C and a further 20 hours of hydrogenation are needed to obtain complete conversion. The apparatus is decompressed and flushed with nitrogen. The reaction mixture is filtered off through Hyflo [91053-39-3] (Hyflo Super Cel medium, Fluka 76063), and the filtrate is concentrated by evaporation. The residue is then concentrated by evaporation repeatedly with acetonitrile (3×20 ml) and then dried under high vacuum. 25.800 mmol (75%) of the title compound are obtained from the residue as a light grey solid. The substance is identical to already published material from WO 2002/040484.

### Example 3A

### Process for Preparing 6,7-dihydro-5H-imidazo[1,5-a]pyridin-8-one (Formula I)

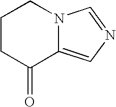

A solution of 0.819 mmol of 5,6,7,8-tetrahydroimidazo[1,5-a]pyridine [38666-30-7] in 3 ml of acetonitrile is admixed with 2.457 mmol of potassium permanganate and stirred vigorously at room temperature over 16 hours. The acetonitrile is removed completely by rotary evaporation, and the residue is taken up in 15 ml of aqueous 0.1M HCl and kept in an ultrasound bath over approx. 2 minutes. The brown suspension is filtered, and the filtercake is washed with 5 ml of aqueous 0.1M HCl. The combined yellow filtrates are basified with aqueous 2M NaOH and extracted with dichloromethane (3×25 ml). The combined organic phases are dried over sodium sulphate and concentrated by evaporation. 0.617 mmol (75% crude yield) of the title compound is obtained from the residue as a yellow oil. The purity of the crude substance is 90% by NMR. The substance is intrinsically identical to already published material from WO 2002/040484.

1. Process for preparing 6,7-dihydro-5H-imidazo[1,5-a]pyridin-8-one (formula I) 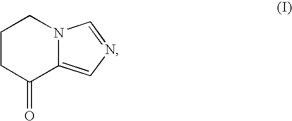
characterized in that
2a) a compound of the formula VIII 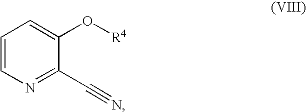
where R⁴ is a suitable protecting group removable by hydrogenation under certain conditions, for example benzyl or 4-methoxybenzyl,
is selectively hydrogenated at the cyano functionality to a compound of the formula IX
or
2a′) 2-hydroxymethylpyridin-3-ol hydrochloride (formula (VIII′) 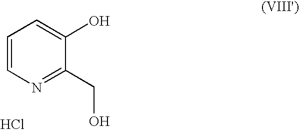
is converted by selective phase transfer-catalysed O-alkylation of the alcohol in the 3 position with an R⁴-halide, followed by mesylation of the primary alcohol, reaction with potassium phthalimide and subsequent treatment with aqueous dimethylamine solution, preferably 40% aqueous dimethylamine solution, to a compound of the formula IX,
2b) the compound of the formula IX 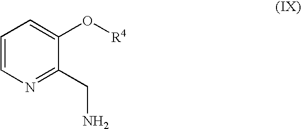
is converted by reaction with formic acid to give the compound of the formula X 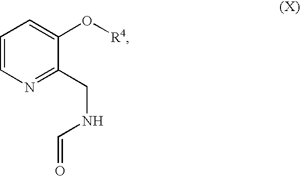
2c) the compound of the formula X is converted to the compound of the formula XI 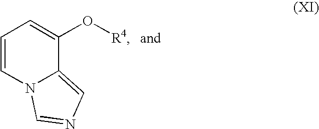
2d) the compound of the formula XI is hydrogenated to obtain 6,7-dihydro-5H-imidazo[1,5-a]pyridin-8-one (formula I).

2. Process for preparing 6,7-dihydro-5H-imidazo[1,5-a]pyridin-8-one (formula I) 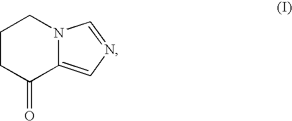
characterized in that
1a) an aldehyde of the formula II 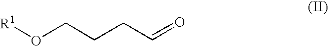
in which R¹ is a suitable protecting group which is either orthogonal to the other protecting groups used in the process, for example benzyl or 4-methoxybenzyl, or which can be concomitantly removed, for example a trialkylsilyl group, is reacted with N,N-dimethyl-2-(trialkylsilanyl)imidazole-1-sulphonamide (formula III) 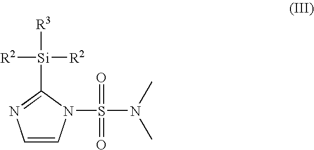
in which R² and R³ are each independently methyl, ethyl, isopropyl, tert-butyl, isobutyl, where
R³ is additionally also —C(CH₃)₂—CH(CH₃)₂,
to give a compound of the formula IV 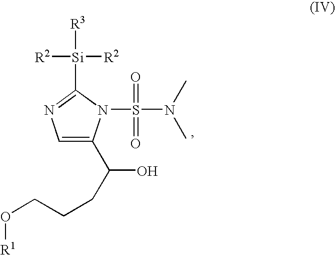
1b) the OH group of the compound of the formula IV is oxidized to a ketone to thus obtain a compound of the formula V 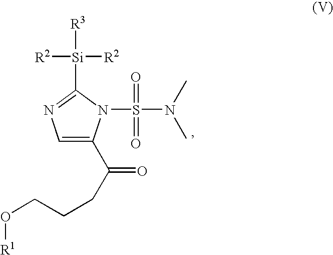
1c) the protecting groups on the imidazole of the compound of the formula V are removed to obtain a compound of the formula VI 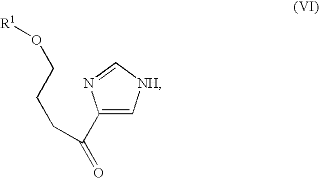
in which
R¹ is a suitable protecting group orthogonal to the other protecting groups used in the process, for example benzyl or 4-methoxybenzyl,
1d) the protecting group of the alcohol function of the compound of the formula VI is removed to thus obtain 4-hydroxy-1-(1H-imidazol-4-yl)butan-1-one (formula VII) 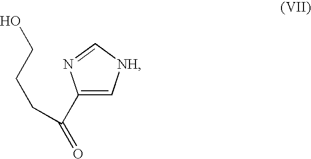
whereby step 1d is obsolete in case the protecting group R¹ is chosen to be a protecting group which can be concomitantly removed together with the protecting groups on the imidazole moiety as 4-hydroxy-1-(1H-imidazol-4-yl)butan-1-one (formula VII) is obtained as the product in step 1c), and
1e) the alcohol function of the 4-hydroxy-1-(1H-imidazol-4-yl)butan-1-one (formula VII) is converted to a leaving group followed by the ring closure to 6,7-dihydro-5H-imidazo[1,5-a]pyridin-8-one (formula I).

3. Process for preparing 6,7-dihydro-5H-imidazo[1,5-a]pyridin-8-one (formula I) 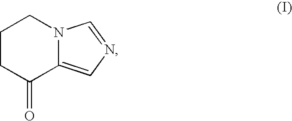
characterized in that
3a) 5,6,7,8-tetrahydroimidazo[1,5-a]pyridine (formula XII) 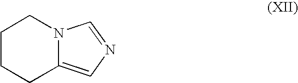
is oxidized regioselectively to obtain 6,7-dihydro-5H-imidazo[1,5-a]pyridin-8-one (formula I).

4. A compound of the formula X 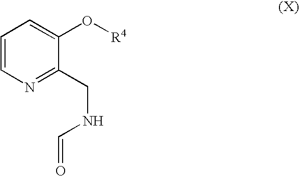
in which R⁴ is a suitable protecting group removable by hydrogenation, for example benzyl or 4-methoxybenzyl.

5. A compound of the formula XI 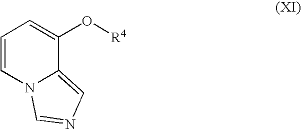
in which R⁴ is a suitable protecting group removable by hydrogenation, for example benzyl or 4-methoxybenzyl.

6. A compound of the formula IV 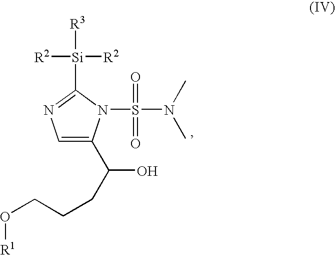
in which
R¹ is a suitable protecting group which is either orthogonal to the other protecting groups used in the process, for example benzyl or 4-methoxybenzyl, or which can be concomitantly removed, for example a trialkylsilyl group, and
R² and R³ are each independently methyl, ethyl, isopropyl, tert-butyl or isobutyl or where R³ is additionally also —C(CH₃)₂—CH(CH₃)₂.

7. A compound of the formula V 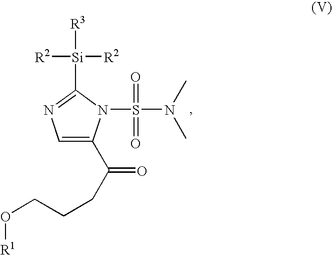
in which
R¹ is a suitable protecting group and is either orthogonal to the other protecting groups used in the process, for example benzyl or 4-methoxybenzyl, or can be concomitantly removed, for example a trialkylsilyl group, and
R² and R³ are each independently methyl, ethyl, isopropyl, tert-butyl or isobutyl or where R³ is additionally also —C(CH₃)₂—CH(CH₃)₂.

8. A compound of the formula VI 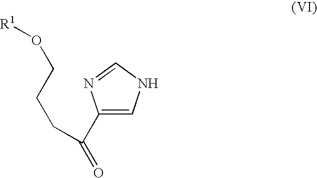
in which
R¹ is a suitable protecting group orthogonal to the other protecting groups used in the process, for example benzyl or 4-methoxybenzyl.
